(19) United States
(12) Patent Application Publication
(10) Pub. No.: US 2004/0161478 A1
(43) Pub. Date: Aug. 19, 2004

(54) Preventives or remedies for arthritis

(76) Inventors: Rysuke Nakagiri, Tokyo (JP); Toshikazu Kamiya, Tsuchiura-shi (JP); Toshio Suda, Sunto-gun (JP); Ichiro Miki, Mishima-shi (JP)

Correspondence Address: FITZPATRICK CELLA HARPER & SCINTO, 30 ROCKEFELLER PLAZA, NEW YORK, NY, 10112, US

(21) Appl. No.: 10/480,044

(22) PCT Filed: Jun. 11, 2002
(86) PCT No.: PCT/JP02/05790

(30) Foreign Application Priority Data
Jun. 15, 2001 (JP) 2001-181947
Mar. 14, 2002 (JP) 2002-70702

(51) Int. Cl. A61K 35/78

(52) U.S. Cl. 424/725

(57) ABSTRACT

The present invention relates to an agent for preventing or treating arthritis which comprises as an active ingredient a plant belonging to the family Saxifragaceae or its extract, foods and drinks or feeds comprising a plant belonging to the family Saxifragaceae or its extract, a method for preventing or treating arthritis in animals which comprises giving animals an agent or a feed for preventing or treating arthritis, and a food and drink or feed for preventing or treating arthritis which comprises as an active ingredient a plant belonging to the family Saxifragaceae or its extract.

### TECHNICAL FIELD

The present invention relates to an agent for preventing or treating arthritis, foods and drinks or feeds having an effect to prevent or treat arthritis, additives for foods and drinks or for feeds having an effect to prevent or treat arthritis, as well as a method for preventing or treating arthritis in animals.

### BACKGROUND ART

Hydrangea macrophylla Seringe var. thunbergii Makino belonging to the family Saxifragaceae is an allied species of hydrangea and said to have been made through breeding of Hydrangea macrophylla SER var. acuminata in the Edo era. Hydrangeae Dulcis Folium is prepared by fermentation of the harvested leaves and branch ends of Hydrangea macrophylla Seringe var. thunbergii Makino, followed by drying.

Hydrangeae Dulcis Folium or its extract has long been used as a corrective (sweetener) in pills or as a raw material for buccal refrigerants. The aqueous extract of Hydrangeae Dulcis Folium has been used as a sweetener in the form of liquid or powder. The sweetening component is phyllodulcin.

Hydrangeae Dulcis Folium has been known to have an anti-coccidium activity, anti-fungal activity, anti-ulcer activity, anti-allergic activity, hypercholes-terolemia-suppressing activity, anti-periodontal bacteria activity, anti-oxidative activity, and the like (the 2nd Symposium on Medicines and Foods, Summary of Lectures, p.85, 1999). The extract of Hydrangeae Dulcis Folium is known to have a cholagogic effect (Yakugaku Zasshi (Journal of Pharmaceutical Society of Japan), vol.114, no.6, 401-413, 1994). In addition, the extract of Hydrangeae Dulcis Folium is also known to show in vitro the effect of inhibiting lipid peroxidation in liver microsomes (Natural Medicines, 49 (1), 84-87, 1995).

Saxifraga stolonifera Meerb. belonging to the family Saxifragaceae is a perennial herb growing in colonies on damp rocks in the mountains. The leaves of Saxifraga stolonifera Meerb. have long been known among Japan and China as one of folk medicines, which are believed to exhibit the effect to a skin burn by sticking thereon after crumpling or drying over a fire. The leaves are sometimes supplied for food.

Saxifraga stolonifera Meerb. or its extract has been used as a cosmetic for whitening, a cosmetic for preventing aging of the skin or as a cosmetic for preventing spots or freckles on the face.

Saxifraga stolonifera Meerb. is also known to have a whitening effect (Japanese Patent No.2749218), effect resisting to the mutation induced by ultraviolet light (Japanese Patent No. 1851704), deodorant effect (Japanese Patent No.2015731), testosterone 5α-reductase inhibitory effect (Japanese Published unexamined Patent Application No.17365/93), anti-oxidative effect (Japanese Published Unexamined Patent Application No. 46142/98), anti-inflammatory effect by inhibition of histamine release (Japanese Published Unexamined Patent Application No.346911/92), and the like.

However, it has not been known that the plants belonging to the family Saxifragaceae such as Hydrangeae Dulcis Folium and Saxifraga stolonifera Meerb. have an effect of prevention or treatment of arthritis.

### DISCLOSURE OF THE INVENTION

An object of the present invention is to provide an agent for preventing or treating arthritis, foods and drinks or feeds having an effect to prevent or treat arthritis, additives for foods and drinks or for feeds having an effect to prevent or treat arthritis, as well as a method for preventing or treating arthritis in animals.

The invention relates to the following (1) to (46).

(1) An agent of preventing or treating arthritis which comprises a plant belonging to the family Saxifragaceae or its extract as an active ingredient.

(2) An agent for preventing or treating arthritis according to the above (1), wherein the plant belonging to the family Saxifragaceae belongs to the genus Saxifraga.

(3) An agent for preventing or treating arthritis according to the above (2), wherein the plant belonging to the genus Saxifraga is Saxifraga stolonifera Meerb.

(4) An agent for preventing or treating arthritis according to the above (1), wherein the plant belonging to the family Saxifragaceae belongs to the genus Hydrangea.

(5) An agent for preventing or treating arthritis according to the above (4), wherein the plant belonging to the genus Hydrangea is Hydrangea macrophylla Seringe var. Thunbergii Makino.

(6) An agent for preventing or treating arthritis according to any of the above (1) to (5), wherein the extract of the plant belonging to the family Saxifragaceae is an alcohol extract of the residue of the extract extracted with an aqueous medium.

(7) An agent for preventing or treating arthritis according to any of the above (1) to (6), wherein the arthritis is chronic articular rheumatism.

(8) An agent for preventing or treating arthritis according to any of the above (1) to (7), which is administered orally.

(9) A food and drink which comprises a plant belonging to the family Saxifragaceae or its extract.

(10) A food and drink according to the above (9), which is used for preventing or treating arthritis.

(11) A food and drink according to the above (9) or (10), wherein the plant belonging to the family Saxifragaceae belongs to the genus Saxifraga.

(12) A food and drink according to the above (11), wherein the plant belonging to the genus Saxifraga is Saxifraga stolonifera Meerb.

(13) A food and drink according to the above (9) or (10), wherein the plant belonging to the family Saxifragaceae belongs to the genus Hydrangea.

(14) A food and drink according to the above (13), wherein the plant belonging to the genus Hydrangea is Hydrangea macrophylla Seringe var. Thunbergii Makino.

(15) A food and drink according to any of the above (9) to (14), wherein the extract of the plant belonging to the family Saxifragaceae is an alcohol extract of the residue of the extract extracted with an aqueous medium.

(16) A feed which comprises a plant belonging to the family Saxifragaceae or its extract.

(17) A feed according to the above (16), which is used for preventing or treating arthritis.

(18) A feed according to the above (16) or (17), wherein the plant belonging to the family Saxifragaceae belongs to the genus Saxifraga.

(19) A feed according to the above (18), wherein the plant belonging to the genus Saxifraga is Saxifraga stolonifera Meerb.

(20) A feed according to the above (16) or (17), wherein the plant belonging to the family Saxifragaceae belongs to the genus Hydrangea.

(21) A feed according to the above (20), wherein the plant belonging to the genus Hydrangea is Hydrangea macrophylla Seringe var. Thunbergii Makino.

(22) A feed according to any of the above (16) to (21), wherein the extract of the plant belonging to the family Saxifragaceae is an alcohol extract of the residue of the extract extracted with an aqueous medium.

(23) An additive for foods and drinks having an effect to prevent or treat arthritis, which comprises a plant belonging to the family Saxifragaceae or its extract.

(24) An additive for foods and drinks according to the above (23), wherein the plant belonging to the family Saxifragaceae belongs to the genus Saxifraga.

(25) An additive for foods and drinks according to the above (24), wherein the plant belonging to the genus Saxifraga is Saxifraga stolonifera Meerb.

(26) An additive for foods and drinks according to the above (23), wherein the plant belonging to the family Saxifragaceae belongs to the genus Hydrangea.

(27) An additive for foods and drinks according to the above (26), wherein the plant belonging to the genus Hydrangea is Hydrangea macrophylla Seringe var. Thunbergii Makino.

(28) An additive for foods and drinks according to any of the above (23) to (27), wherein the extract of the plant belonging to the family Saxifragaceae is an alcohol extract of the residue of the extract extracted with an aqueous medium.

(29) An additive for feeds having an effect to prevent or treat arthritis, which comprises a plant belonging to the family Saxifragaceae or its extract.

(30) An additive for feeds according to the above (29), wherein the plant belonging to the family Saxifragaceae belongs to the genus Saxifraga.

(31) An additive for feeds according to the above (30), wherein the plant belonging to the genus Saxifraga is Saxifraga stolonifera Meerb.

(32) An additive for feeds according to the above (29), wherein the plant belonging to the family Saxifragaceae belongs to the genus Hydrangea.

(33) An additive for feeds according to the above (32), wherein the plant belonging to the genus Hydrangea is Hydrangea macrophylla Seringe var. Thunbergii Makino.

(34) An additive for feeds according to any of the above (29) to (33), wherein the extract of the plant belonging to the family Saxifragaceae is an alcohol extract of the residue of the extract extracted with an aqueous medium.

(35) A method for preventing or treating arthritis in animals which comprises administering an agent for preventing or treating arthritis according to any of the above (1) to (7) to an animal or feeding a feed according to any of the above (16) to (22) to an animal.

(36) A method according to the above (35), wherein the animal is a pet or domestic animal.

(37) A food and drink or feed for preventing or treating arthritis, which comprises a plant belonging to the family Saxifragaceae or its extract as an active ingredient.

(38) A food and drink or feed for preventing or treating arthritis according to the above (37), wherein the plant belonging to the family Saxifragaceae belongs to the genus Saxifraga.

(39) A food and drink or feed for preventing or treating arthritis according to the above (38), wherein the plant belonging to the genus Saxifraga is Saxifraga stolonifera Meerb.

(40) A food and drink or feed for preventing or treating arthritis according to the above (37), wherein the plant belonging to the family Saxifragaceae belongs to the genus Hydrangea.

(41) A food and drink or feed for preventing or treating arthritis according to the above (40), wherein the plant belonging to the genus Hydrangea is Hydrangea macrophylla Seringe var. Thunbergii Makino.

(42) A food and drink or feed for preventing or treating arthritis according to any of the above (37) to (41), wherein the extract of the plant belonging to the family Saxifragaceae is an alcohol extract of the residue of the extract extracted with an aqueous medium.

(43) Use of a plant belonging to the family Saxifragaceae or its extract for the production of an agent for preventing or treating arthritis.

(44) Use of a plant belonging to the family Saxifragaceae or its extract for the production of a food and drink or feed for preventing or treating arthritis.

(45) Use of a plant belonging to the family Saxifragaceae or its extract for the production of an additive for a food and drink or feed for preventing or treating arthritis.

(46) A method for preventing or treating arthritis which comprises administering a plant belonging to the family Saxifragaceae or its extract.

### BEST MODE FOR CARRYING OUT THE INVENTION

In the present invention, the plants belonging to the family Saxifragaceae mean plants classified under the scientific name Saxifragaceae [Makino's New Illustrated Flora of Japan (40th edition, 1984, Hokuryukan); Makino's Illustrated Flora in Color, Sequel (First edition, 1983, Hokuryukan)].

The plants belonging to the family Saxifragaceae include plants of the genera Astilbe, Rodgersia, Saxifraga, Tanakaea, Aceriphyllum, Boykinia, Chrysosplenium, Tiarell, Mitella, Parnassia, Philadelphus, Deutzia, Platycrater, Hydrangea, Schizophragma, Cardiandra, Deinanthe, Itea, Ribes and Kirengeshoma, and the plants varieties bred from these plants. Preferred are plants derived from Hydrangea or Saxifraga and those bred therefrom which have enhanced protective or therapeutic effect for arthritis.

Examples of the plants belonging to the genus Astilbe are Astilbe microphylla Knoll, Astilbe thunbergii Miq., Astilbe odontophylla Miq., Astilbe japonica Miq. and Astilbe simplicifolia Makino.

An example of the plants belonging to the genus Rodgersia is Rodgersia podophylla A. Gray.

Examples of the plants belonging to the genus Saxifraga are Saxifraga stolonifera Meerb., Saxifraga nipponica Makino, Saxifraga fortunei Hook, Saxifraga cortusaefolia Sieb. et Zucc., Saxifraga japonica Boiss., Saxifraga fusca Maxim, Saxifraga sendaica Maxim. var. laciniata Nakai, Saxifraga merkii Fisch. var. idsuroei Engl., Saxifraga laciniata Nakai et Takeda, Saxifraga bronchialalis L., Saxifraga cernua L. and Saxifraga sachalinensis Fr. Schm. Saxifraga stolonifera Meerb. is preferred among them.

An example of the plants belonging to the genus Tanakaea is Tanakaea radicans Franch. et Sav.

An example of the plants belonging to the genus Aceriphyllum is Aceriphyllum rossii Engler.

Examples of the plants belonging to the genus Boykinia are Boykinia lycoctonifolia Engl. and Boykinia tellimoides Engl. et Irmsch. Examples of the plants belonging to the genus Chrysosplenium are Chrysosplenium grayanum Maxim., Chrysosplenium stamineum Franch, Chrysosplenium japonicum Makino, Chrysosplenium flagelliferum Fr.Schm., Chrysosplenium macrostemon Maxim. and Chrysosplenium sphaerospermum Maxim.

An example of the plants belonging to the genus Tiarell is Tiarella polyphylla Don.

Examples of the plants belonging to the genus Mitella are Mitella japonica Miq. and Mitella pauciflora Rosend.

Examples of the plants belonging to the genus Parnassia are Parnassia palustris L., Parnassia alpicola Makino and Parnassia foliosa Hook.f. et Thoms. var. nummularia Nakai.

An example of the plants belonging to the genus Philadelphus is Philadelphus satsumi Sieb.

Examples of the plants belonging to the genus Deutzia are Deutzia crenata Sieb. et Zucc., Deutzia sieboldiana Maxim., Deutzia gracilis Sieb. et. Zucc., Deutzia maximowicziana Makino, Deutzia uniflora Shirai and Deutzia gracilis Sieb. et Zucc., var. nagurai Makino.

An example of the plants belonging to the genus Platycrater is Platycrater serrata Makino.

Examples of the plants belonging to the genus Hydrangea are Hydrangea macrophylla Seringe, Hydrangea macrophylla Seringe var. otaksa Makino, Hydrangea macrophylla Seringe subsp. serrata Makino var. japonica Makino, Hydrangea macrophylla Seringe var. acuminata, Hydrangea macrophylla Seringe var. Thunbergii Makino, Hydrangea scandens Seringe, Hydrangea hirta Sieb. et Zucc., Hydrangea involucrata Sieb., Hydrangea sikokiana Maxim., Hydrangea paniculata Sieb., Hydrangea petiolaris Sieb. et Zucc., Hydrangea macrophylla Seringe subsp. serrata Makino var. amoena Makino and Hydrangea macrophylla Seringe subsp. serrata Makino var. angustata Makino. Hydrangea macrophylla Seringe var. Thunbergii Makino is preferred among them.

An example of the plants belonging to the genus, Schizophragma is Schizophragma hydrangeoides Sieb. et Zucc.

An example of the plants belonging to the genus Cardiandra is Cardiandra alternifolia Sieb. et Zucc.

An example of the plants belonging to the genus Deinanthe is Deinanthe bifida Maxim.

An example of the plants belonging to the genus Itea is Itea japonica Oliver.

Examples of the plants belonging to the genus Ribes are Ribes sinanense F. Maekawa, Ribes grossularia L., Ribes latifolium Jancz., Ribes rubrum L., Ribes japonicum Maxim., Ribes sachalinense Nakai, Ribes fasciculatum Sieb. et Zucc., Ribes alpinum L. var. japonicum Maxim. and Ribes ambiguum Maxim.

An example of the plants belonging to the genus Kirengeshoma is Kirengeshoma palmata Yatabe.

In the present invention, the term “plant” also includes leaves, flowers, branches, stems, fruits, roots, seeds, cultured cells, organs or callus of wild plants, cultivated plants, or plants obtained by culturing such as tissue culture, which are used as such or after being treated physically, chemically or biologically.

The physical or chemical treatment includes drying such as sun-drying, air-drying, and freeze-drying, and disruption. The physically or chemically treated matters include dried matters, freeze-dried matters and disrupted matters. The biological treatment includes fermentation, and the biologically treated matters include fermented matters.

As the plant used in the present invention, the fermented matters obtained by fermentation is preferred, particularly Hydrangeae Dulcis Folium obtained by fermentation of Hydrangea macrophylla Seringe var. thunbergii Makino is preferred.

The extracts of plants include those obtained from the above-described plants by various methods of extraction. The method of extraction includes extraction with various solvents and supercritical fluid extraction. The extract may further be treated by various methods of solid-liquid separation such as sedimentation, cake filtration, clear filtration, centrifugal filtration, centrifugal sedimentation, compression separation and filter press, various concentration methods, various drying methods, methods of making various preparations such as granulation or pulverization, and various purification methods. The purification methods include solvent fractionation, column chromatography and recrystallization.

Particularly, the column chromatography using various carriers such as DIAION HP-20 (Mitsubishi Chemical Corporation) and Sephadex LH-20 (Pharmacia) is preferred.

Examples of the concentration and drying methods are freeze-drying, natural-drying, hot air-drying, air-drying, drying by blowing, spray drying, drying under reduced pressure, sun-drying, vacuum drying, fluidized-bed drying, foam-bed drying, film drying with a drum dryer, ultrasonic drying and electromagnetic ware drying, and the preferred methods are spray drying and freeze-drying.

In the step of extraction and treatment of the extract, an anti-oxidant, preservative, etc. may be added.

As the solvent for extraction, any type of solvent, which can extract a substance having a protective or therapeutical effect for arthritis, may be used. Suitable solvents include aqueous media such as water, an aqueous solution of inorganic salt, buffer solution; monovalent alcohols such as methanol, ethanol, propanol and butanol; polyvalent alcohols such as propylene glycol and glycerol; and organic solvents such as hexane, toluene, petroleum ether, benzene, ethyl acetate, chloroform, dichloromethane, 1,1,2-trichloroethene, dimethylsulfoxide and acetone; and preferred are the aqueous media and alcohols.

Examples of the buffers are phosphate buffer and citrate buffer. The inorganic salt used in the aqueous solution of inorganic salt includes sodium chloride, potassium chloride and calcium chloride.

The preferred alcohols are monovalent alcohols, and a preferred monovalent alcohol is ethanol.

Water may be tap water, distilled water, deionized water, or pure water.

In extracting an active ingredient from the plant containing the active ingredient, it is preferable to use a solvent which is usable for foods and drinks or feeds, for example, water, water-containing ethanol, or anhydrous ethanol, and the like.

These solvent may be used alone or as a mixture. As the mixed solvent, water-containing alcohols are preferred. More preferred are monovalent alcohols, and water-containing ethanol is specifically preferred. The water content is preferably 70% or less, more preferably 40% or less.

As the solvent, supercritical fluid carbon dioxide may also be used.

For extraction, the solvent may be used, for example, in an amount of 0.1 to 10,000 parts by weight preferably 1 to 100 parts by weight for 1 part by weight of a plant. There is no particular limitation for the extraction temperature, but the extraction is preferably carried out at 0 to 100° C., more preferably 20 to 90° C. There is no particular limitation for the extraction time, but it is preferably 1 minute to 1 week, more preferably 30 minutes to 1 day.

The extract containing the active ingredient may be prepared by extraction with various solvents or by supercritical fluid extraction under the condition that the active ingredient can be extracted from the plant containing the active ingredient. The extract thus obtained may further be subjected to extraction with various solvents or by supercritical fluid extraction under the condition that the active ingredient can be extracted. When the active ingredient from the plant cannot be extracted or hardly extracted by extraction with various solvents or by supercritical fluid extraction, the residue obtained after the extraction may further be subjected to extraction with various solvents or by supercritical fluid extraction under the condition that the active ingredient can be extracted.

Specifically, extraction from the plant belonging to the genus Hydrangea is preferably carried out by subjecting the plant per se or physically, chemically and/or biologically treated matter of the plant to extraction with an aqueous medium, and then by subjecting the resulting residue (hereinafter referred to as the extraction residue with aqueous medium) to extraction with an alcohol or water-containing alcohol or acetone.

There is no particular limitation for the aqueous medium, but water is preferred. The extraction with an aqueous medium, alcohol, water-containing alcohol or acetone is carried out preferably at a temperature of 0 to 100° C., more preferably 20 to 90° C. There is no particular limitation for the extraction time, but it is preferably 1 minute to 1 week, more preferably 30 minutes to 1 day. It is preferred to the plants of the family Saxifrage after drying. The extraction residue with aqueous medium may be prepared by extracting the plant of the family Saxifrage with an aqueous medium, followed by removal of the aqueous medium through filtration, etc.

There is no particular limitation as to the apparatus used for extraction, and preferred are a vessel designed for efficient extraction, a stirrer, a reflux condenser, a Soxhlet extractor, a homogenizer, a shaker, an ultrasonic generator etc.

There is no limitation as to the arthritis to which the present invention can be applied, and examples include chlamydial arthritis, chronic absorptive arthritis, enteropathic arthritis, gonococcal arthritis, gouty arthritis, Jaccoud's arthritis, juvenile arthritis, Lyme arthritis, ochronotic arthritis, suppurative arthritis, osteoarthritis, shoulder periarthritis, arthritis caused by hyperkinesis, chronic articular rheumatism. The present invention is particularly effective for chronic articular rheumatism.

The followings will illustrate agents for preventing or treating arthritis, foods and drinks, animal feeds, food additives and feed additives of the present invention.

(1) An Agent for Preventing or Treating Arthritis Which Comprises a Plant Belonging to the Family Saxifragaceae or its Extract as an Active Ingredient

An agent for preventing or treating arthritis of the present invention, which comprises the plant belonging to the family Saxifragaceae or its extract as an active ingredient, contains a plant belonging to the family Saxifragaceae or its extract prepared in the manner mentioned above, and it may further contain one or more kinds of pharmacologically acceptable carriers and another active ingredient for prevention or treatment, if necessary. In addition, the agent of the present invention for preventing or treating arthritis may contain any other ingredient effective for preventing or treating arthritis.

Examples of other ingredients effective for preventing or treating arthritis (hereinafter sometimes simply referred to as other effective ingredients) include boron, calcium, chromium, copper, magnesium, manganese, selenium, silicon, zinc, S-adenosylmethionine, collagen, collagen hydrolyzate, gelatin, gelatin hydrolyzate, bromelain, trypsin, chymotrypsin, papain, rutin, carotenoid, flavonoid, anti-oxidant vitamin, γ-linolenic acid, eicosapentaenoic acid, Boswellia, capsaicin, cat's claw, devil's claw, feverfew, ginger, nettles, niacinamide, shark ointment, turmeric and curcumin. These ingredients may be in pure forms or as a mixture containing them or as an extract.

An agent for preventing or treating arthritis of the present invention can be produced by mixing a plant belonging to the family Saxifragaceae or its extract with one or more pharmacologically acceptable carriers, if required together with another active ingredient, in a way conventionally used in the field of pharmaceutical technology.

It is desirable to administer an agent for preventing or treating arthritis of the present invention through an effective route for preventing or treating arthritis, for example, by oral administration or perenteral administration such as intravenous administration.

The dosage form includes, for example, tablets, powders, granules, pills, suspensions, emulsions, infusions, capsules, syrups, injections, liquid preparations, elixirs, extracts, tinctures, fluid extracts, and the like.

The formulation suitable for oral administration, e.g., extracts, tinctures or fluid extracts, may be prepared by extracting a plant of the family Saxifragaceae with water, ethanol or a mixture of water and ethanol, etc., if necessary followed by concentration.

The liquid formulation suitable for oral administration, e.g., syrups, may be prepared using carriers, such as water, a sugar (e.g., sucrose, sorbitol or fructose), a glycol (e.g., polyethylene glycol or propylene glycol), or an oil (e.g., sesame oil, olive oil or soybean oil); an antiseptic (e.g., p-hydroxybenzoate); a preservative such as paraoxybenzoate derivatives (e.g., methyl paraoxybenzoate), sodium benzoate; and a perfume such as strawberry flavor or peppermint.

Tablets., powders, granules, etc., which are suitable for oral administration, may be prepared using a sugar such as lactose, white sugar, glucose, sucrose, mannitol or sorbitol; starch such as potato starch, wheat starch or corn starch; an inorganic substance such as calcium carbonate, calcium sulfate, sodium hydrogen carbonate or sodium chloride; an excipient such as crystalline cellulose or plant powder (e.g., licorice powder or gentian powder); a disintegrator such as starch, agar, gelatin powder, crystalline cellulose, carmellose sodium, carmellose calcium, calcium carbonate, sodium hydrogen carbonate or sodium alginate; a lubricant such as magnesium stearate, talc, hydrogenated vegetable oil, macrogol or silicone oil; a binder such as polyvinyl alcohol, hydroxypropyl cellulose, methylcellulose, ethylcellulose, carmellose, gelatin or starch paste; a surfactant such as fatty acid ester; and/or a plasticizer such as glycerin.

The formulation suitable for non-oral administration preferably comprises a sterilized aqueous preparation containing an active compound which is isotonic to the recipient's blood. For example, for injections, an injectable solution maybe prepared using a carrier such as a salt solution, a glucose solution, or a mixture of a salt solution and a glucose solution.

In these non-oral preparations, it is possible to add one or more of supplementary components for the oral preparations mentioned above, selected from carriers, antiseptics, preservatives, flavors, excipients, disintegrators, lubricants, binders, surfactants, and plasticizers.

The dose and frequency of the administration of the agent for preventing or treating arthritis of the present invention depend on the dosage form, age of the patient, body weight, and the symptom and degree of the disease to be treated. For example, in oral administration, a plant of the family Saxifragaceae or its extract may be administered preferably in an amount of 1 mg to 50 g by dry weight, more preferably 5 mg to 10 g, once or several times a day for an adult. In non-oral administration, e.g., intravenous administration, a plant of the family Saxifragaceae or its extract may be administered preferably in an amount of 0.001 to 50 g by dry weight, more preferably 0.01 to 10 g, once or several times a day for an adult.

Arthritis can be prevented by daily administering an agent for arthritis of the present invention. In the case already attacked by arthritis, it is possible to alleviate or cure the arthritis by daily administering an agent of arthritis of the present invention. An agent for preventing or treating arthritis of the present invention is administered usually for a period of 1 week to 10 years, preferably 1 month to 5 years.

The agent for preventing or treating arthritis of the present invention can be used not only for humans but also for animals other than humans. There is no particular limitation as to the dosage for use in animals. For examples, a plant belonging to the family Saxifragaceae or its extract is administered preferably at a dose of 0.01 to 50 g/kg, and more preferably 0.05 to 10 g/kg by dry weight.

(2) A Food and Drink or Feed for Preventing or Treating Arthritis Which Comprises a Plant Belonging to the Family Saxifragaceae or its Extract as an Active Ingredient

As the food and drink or feed for preventing or treating arthritis of the present invention which comprises a plant belonging to the family Saxifragaceae or its extract as an active ingredient, a plant belonging to the family Saxifragaceae or the extract extracted therefrom in a method as mentioned above is used as such as the food and drink or feed; or alternatively the plant or its extract is used as additives to foods and drinks or feeds. Thus, the food and drink or feed can be used as health food and drink, functional food and drink, etc., for the purpose of preventing or treating arthritis.

(3) A Food and Drink or Feed to Which a Plant Belonging to the Family Saxifragaceae or its Extract is Added

The food and drink or feed of the present invention, to which a plant belonging to the family Saxifragaceae or its extract is added, may be produced by adding a plant of the family Saxifragaceae or the extract extracted therefrom in a method as mentioned above to a food and drink or feed which does not contain the plant or its extract.

The food and drink or feed of the present invention may be produced in a conventional way for production of usual foods and drinks or feeds, except that a plant of the family Saxifragaceae or its extract and if necessary other active ingredients are added.

The food and drink or feed thus obtained may be processed according to a method for processing the usual foods and drinks or feeds, for example, molding or granulation. Examples of the processing method includes granulation methods such as fluid bed granulation, stirring granulation, extrusion granulation, oscillating granulation, gas stream granulation, compression molding granulation, disruption granulation, spray granulation, and jet granulation; coating methods such as pan coating, fluid bed coating and dry coating; and imbibition methods such as puff drying, excess steam method, foam mat method and microwave heating method.

There is no particular limitation for the amount of a plant belonging to the family Saxifragaceae or its extract to be added as far as the foods and drinks or feeds of the present invention has an effect to prevent or treat arthritis, and the amount may be, for example, 0.01 to 50% by weight, preferably 0.1 to 10% by weight.

Examples of the foods and drinks prepared by adding a plant belonging to the family Saxifragaceae or its extract include health foods, health drinks, juice, soft drinks, soup, tea, dairy products such as lactic acid bacterium drinks, fermented milk, ice cream, butter, cheese, yogurt, processed milk and skim milk, meat products such as ham, sausage and hamburger, fish paste foods, egg products such as Dashimaki (omelet with stock) and Tamago-dofu (steamed beaten egg with soup stock), confectionary such as cookies, jelly, snacks and chewing gum, bread, noodles, pickles, smoked fish and meat, dry fish, Tsukudani (simmered meat in soy sauce and sugar), and seasonings, to all of which a plant belonging to the family Saxifragaceae or its extract is added.

There is no particular limitation as to the form of the foods and drinks as far as they contain a plant belonging to the family Saxifragaceae or its extract, and examples include powdered foods, sheet-shaped foods, bottled foods, canned foods, retort foods, capsule foods, tablet foods, liquid foods, drinkable preparations, and the like.

To the foods and drinks of the present invention, if required, it is possible to add one or more additives generally used in foods and drinks, for example, sweetener, coloring agent, preservatives, thickening stabilizer, antioxidant, color-developing agent, bleaching agent, anti-fungal agent, gum base, bitter agents, enzymes, wax, sour agent, seasonings, emulsifier, nutrient supplements, additional materials for preparation, flavors, spice extracts, and the like.

Examples of the sweeteners are aspartame, licorice, stevia, xylose and Momordica grosvenori.

Examples of the coloring agent are carotenoid or turmeric pigment, flavonoid, caramel pigment, oriental gromurell pigment, spirulina pigment, chlorophyll, red sweet potato pigment, red Chinese yam pigment, perilla pigment and blueberry pigment.

Examples of the preservatives are sodium sulfite, benzoic acids, extract of Aralia cordata, extract of Styrax japonica, extract of Artemisia capillaris, sorbic acids, propionic acids, and the like.

Examples of the thickening stabilizers are gums such as gum arabic or xanthan gum, alginic acids, chitin, chitosan, aloe extract, guar gum, hydroxypropylcellulose, casein sodium, corn starch, carboxymethylcelluloses, gelatin, agar, dextrin, methylcellulose, polyvinyl alcohol, microfibrous cellulose, microcrystalline cellulose, seaweed cellulose, sodium polyacrylate, sodium polyphosphate, carrageenan, yeast cell wall, konjak extract, nata de coco and mannan.

Examples of the antioxidants are vitamin C, sodium ethylenediaminetetraacetate, calcium ethylenediaminetetraacetate, erythorbic acid, oryzanol, catechin, quercetin, clove extract, enzyme-treated rutin, apple extract, sesame oil extract, dibutylhydroxytoluene, fennel extract, horseradish extract, dropwort extract, tea extract, Tempeh extract, extract of Houttuynia cordata, tocotrienol, tocopherols, rapeseed oil extract, green coffee extract, sunflower seed, ferulic acid, butylhydroxyanisole, extract of blueberry leaves, propolis extract, hego-ginkgo extract, hesperetin, pepper extract, garden balsam extract, gallic acid, myrica extract, eucalyptus extract and rosemary extract.

An example of the color-developing agent is sodium sulfite.

An example of the bleaching agent is sodium sulfite.

An example of the anti-fungal agent is o-phenylphenol.

Examples of the gum bases are methyl acetylricinoleate, Japanese lacquer wax, ester gum, elemi resin, urucury wax, ozokerite, opopanax resin, kauri gum, carnauba wax, guaiacum resin, gutta katiau, gutta hangkang, gutta-percha, glycerin fatty acid ester, spermaceti, copaiba balsam, copal resin, gum, rice branwax, sugarcane wax, shellac, jelutong, sucrose fatty acid ester, sorba, sorbitan fatty acid esters, talc, calcium carbonate, dammar resin, chicle, chilte, tunu, low molecular gum, paraffinwax, firbalsam, propylene glycol fattyacidester, powdered pulp, powdered rice husks, jojobawax, polyisobutylene, polybutene, microcrystalline wax, mastic, massaranduba chocolate, beeswax and calcium phosphate.

Examples of the bitter agents are isoalpha bitter acid, caffeine, kawaratake extract, cinchona extract, Amur cork extract, gentian extract, spide extract, enzyme-treated naringin, Jamaica quassia extract, theobromine, naringin, bitter ash extract, extract of Artemisia absinthium, isodonis extract, Himematsutake extract, borapet, methylthioadenosine, litchi extract, olive tea, sour orange extract, hop extract and mugwort extract.

Examples of the enzymes are amylase, trypsin, rennet and lactic acid bacteria.

Examples of the wax are Japanese lacquer wax and vegetable wax.

Examples of the sour agents are adipic acid, itaconic acid, citric acids and citrates, succinic acids and succinates, sodium acetate, tartaric acids and tartrates, carbon dioxide, lactic acid, phytic acid, fumaric acid, malic acid and phosphoric acid.

Examples of the seasonings are amino acids such as asparagine, aspartic acids, glutamic acid, glutamine, alanine, isoleucine, glycine, serine, cystine, tyrosine, leucine, or proline; nucleic acids such as sodium inosinate, sodium uridylate, sodium guanylate, sodium cytidylate, calcium ribonucleotide, or sodium ribonucleotide; organic acids such as citric acid or succinic acid; potassium chloride, sodium solution of low salt content prepared from salt lake water, crude potassium chloride from seawater, whey salt, tripotassium phosphate, dipotassium hydrogenphosphate, potassium dihydrogenphosphate, disodium hydrogenphosphate, sodium dihydrogenphosphate, trisodium phosphate and chlorella extract.

Examples of the emulsifying agents are fatty acid monoglyceride and sorbitan fatty acid ester.

Examples of the nutrient supplements are zinc salts, vitamin C, various amino acids, 5-adenylic acid, iron chloride, hesperidin, various kind of burnt calcium, various kind of unburnt calcium, dibenzoylthiamine, calciumhydroxide, calcium carbonate, thiamine hydrochloride, dunaliella carotene, tocopherol, nicotinic acid, carrot carotene, palmoil carotene, calcium pantothenate, vitamin A, hydroxyproline, calcium dihydrogenpyrophosphate, iron(II) pyrophosphate, iron(III) pyrophosphate, ferritin, heme iron, menaquinone, folic acid and riboflavin.

Examples of the additional materials for preparation are processing aids such as acetone or ion exchange resin, extract of fig leaves, extract of rice straw ash, kaolin, glycerin fatty acid ester, mulberry extract, bone ash, perilla extract, ginger extract, various tannins, Phaffia extract, grape seed extract and ethanol.

Examples of the flavors are those as mentioned above.

Examples of the spice extracts are capsicum extract and garlic extract.

There is no particular limitation as to the intake of the foods and drinks for preventing or treating arthritis which contain a plant belonging to the family Saxifragaceae or its extract or the intake of the foods and drinks prepared by adding thereto a plant belonging to the family Saxifragaceae or its extract, however the amount of the plant to be taken is preferably 0.1 g to 50 g, and more preferably 0.5 g to 10 g by dry weight, a day for an adult preferably for a period of one day to one year, more preferably 2 weeks to 3 months. This intake is merely a typical example and the intake can be appropriately adjusted depending on the age, the condition, and body weight of the recipient.

The foods and drinks may be taken once a day or divided in several times. Daily intake of the foods and drinks is effective in prevention of arthritis. Even if the arthritis has already occurred, the daily intake of the foods and drinks would be effective to alleviate or cure arthritis.

The feeds of the present invention to which has been added a plant belonging to the family Saxifragaceae or its extract can be used as a feed for animals including mammals, birds, reptiles, amphibians and fishes, preferably for mammals. For example, the feeds of the present invention can be used for pets such as dog, cat or mouse, livestocks such as cattle or pig, poultry such as chicken or turkey, or cultivated fishes such as sea bream or young yellowtail, and preferably for pets or livestocks.

The feeds of the present invention may be produced by properly mixing feed materials and a plant belonging to the family Saxifragaceae or its extract. The feed materials include cereals, chaff and bran, vegetable oil cakes, animal feed materials, other feed materials and purified products.

Examples of the cereals are milo, wheat, barley, oats, rye, brown rice, buckwheat, foxtail millet, broomcorn mellet, Japanese millet, corn and soybean.

Examples of the chaff and bran are rice bran, defatted rice bran, wheat bran, wheat middlings, wheat, wheat germ, barely bran, screening, pellets, corn bran and corn germ.

Examples of the vegetable oil cakes are soybean oil cake, soybean flour, linseed oil cake, cotton seed oil cake, peanut oil cake, safflower oil cake, coconut oil cake, palm oil cake, sesame oil cake, sunflower oil cake, rapeseed oil cake, kapok oil cake and mustard seed oil cake.

Examples of the animal feed materials are fish meal (e.g., northern ocean meal, imported meal, whole meal and coastal meal), fish soluble, meat meal, meat and bone meal, blood powder, degraded hair, bone meal, processed by-product for livestocks, feather meal, silk-worm pupa, skim milk, casein and dry whey.

Examples of the other feed materials are stems and leaves of plants (e.g., alfalfa, hay cube, alfalfa leaf meal and the powder of false acacia), processed industrial by-products of corn (e.g., corn gluten, meal, corn gluten feed and corn steep liquor), processed starch product, sugar, fermentation industrial products (e.g., yeast, beer cake, malt root, alcohol cake and soy source cake), agricultural by-products (e.g., processed citrus fruit cake, tofu cake, coffee cake and cocoa cake), and others (e.g. cassava, broad bean, guar meal, sea weed, krill, spirulina, chlorella, minerals).

Examples of the purified products are proteins (e.g., casein and albumin), amino acids, sugars (e.g., starch, cellulose, sucrose and glucose), minerals and vitamins.

There is no particular limitation as to the intake of the feeds for preventing or treating arthritis which contains as an active ingredient a plant belonging to the family Saxifragaceae or its extract or the intake of the feeds prepared by adding a plant belonging to the family Saxifragaceae or its extract, however, it is preferable to take the plant in an amount of 0.01 g to 50 g, more preferably in an amount of 0.05 g to 10 g by dry weight/kg by body weight of an animal per day for a period of 1 week to 5 years, more preferably 2 weeks to 2 years. This intake is merely a typical example, and the intake may be appropriately adjusted depending on the species, age, and body weight of the subject animal.

Daily feeding of the animal feeds of the present invention is effective in prevention of arthritis.

Even if the arthritis has already occurred, the daily feeding of the feeds would be effective to alleviate or cure arthritis.

(4) Additives for Foods and Drinks or Feeds Effective for Preventing or Treating Arthritis Which Comprise a Plant Belonging to the Family Saxifragaceae or its Extract

The additives for foods and drinks or feeds of the present invention having an effect for preventing or treating arthritis which comprise a plant belonging to the family Saxifragaceae or its extract contain as an active ingredient a plant belonging to the family Saxifragaceae or its extract prepared as mentioned above, and may further contain, if necessary, one or more additives for foods and drinks as listed above, for example, sweeteners, coloring agents, preservatives, thickening stabilizers, antioxidants, color-developing agents, bleaching agents, anti-fungal agents, gum bases, bitter agents, enzymes, wax, sour agents, seasonings, emulsifiers, nutrient supplements, additional materials for preparation, flavors and spice extracts. In addition, the carriers as mentioned above may be added.

There is no limitation as to the intake of the additives for foods and drinks of the present invention having an effect to prevent or treat arthritis, however, it is preferable to take a plant belonging to the family Saxifragaceae or an extract thereof in an amount of 0.1 g to 50 g, more preferably 0.5 g to 10 g by dry weight a day for an adult for a period of 1 week to 10 years, more preferably 1 month to 5 years. This intake is merely a typical example, and the intake maybe appropriately adjusted depending on the condition, age, and body weight of the recipient.

There is no limitation as to the amount of the additive of the present invention to be added to foods and drinks, and for example, the amount is 0.01 to 50% by weight, preferably 0.1 to 10% by weight, though there in no particular limitation therein.

The additives for food and drink of the present invention may be added to foods and drinks, and the foods or drinks is taken once a day or divided into several times. Daily intake of the additives for food and drink of the present invention is effective in prevention of arthritis. Even if the arthritis has already occurred, the daily intake of the additives for food and drink would be effective to alleviate or cure arthritis.

There is no limitation as to the intake of the feed additives of the present invention having an effect to prevent or treat arthritis which contains a plant belonging to the family Saxifragaceae or its extract, however, it is preferable to take the plant or its extract in an amount of 0.01 g to 50 g, more preferably 0.05 to 10 g by dry weight per kg by body weight of an animal per day for a period of 1 week to 5 years, more preferably 2 weeks to 2 years. This intake is merely a typical example, and the intake may be appropriately adjusted depending on the species, age, and body weight of the subject animal.

The feed additives of the present invention may be added to feeds and daily fed to animals to prevent arthritis. Even if the arthritis has already occurred, the daily feeding of the feed additives of the present invention added to feeds would be effective to cure arthritis.

There is no limitation as to the amount of the feed additive of the present invention to be added to feeds, however, the amount is for example 0.01 to 50% by weight, preferably 0.1 to 10% by weight.

The following examples will explain the present invention in more detail, but they are not intended to limit the scope of the present invention.

### EXAMPLE 1

Production of Freeze-Dried Powder From a Water Extract of Hydrangeae Dulcis Folium

Dried powder of Hydrangeae Dulcis Folium (1 kg, Shihira Shoten) was extracted twice with 10 liters of distilled water at room temperature with stirring for 1 hour. The resulting extract was concentrated and freeze-dried to give 200 g of freeze-dried powder of Hydrangeae Dulcis Folium from the water extract.

### EXAMPLE 2

Production of Freeze-Dried Powder From a 60% Ethanol-Water Extract of Hydrangeae Dulcis Folium

Dried powder of Hydrangeae Dulcis Folium (1 kg) was extracted twice with 10 liters of 60% aqueous solution of ethanol at room temperature with stirring for 1 hour. The resulting extract was concentrated and freeze-dried to give 200 g of freeze-dried powder of Hydrangeae Dulcis Folium from the 60% ethanol-water extract.

### EXAMPLE 3

Production of Freeze-Dried Powder of Hydrangeae Dulcis Folium From an Acetone Extract

Dried powder of Hydrangeae Dulcis Folium (1 kg) was extracted twice with 10 liters of acetone at room temperature with stirring for 1 hour. The resulting extract was concentrated and freeze-dried to give 100 g of freeze-dried powder of Hydrangeae Dulcis Folium from the acetone extract.

### EXAMPLE 4

Production of Freeze-Dried Powder From a Hot Water Extract of Hydrangeae Dulcis Folium

Dried powder of Hydrangeae Dulcis Folium (1 kg) was extracted with 10 liters of boiling distilled water for 30 minutes. The resulting extract was concentrated and freeze-dried to give 130 g of freeze-dried powder of Hydrangeae Dulcis Folium from the hot water extract.

### EXAMPLE 5

Production of Freeze-Dried Powder From an Ethanol Extract of Hydrangeae Dulcis Folium Water-Extract Residue

Dried powder of Hydrangeae Dulcis Folium (1 kg) was extracted with 20 liters of distilled water at 40° C. with stirring until the absorbance of the 1/200 dilution at 313 nm became 0.15. The obtained extract was filtered to remove the filtrate. The resulting extract residue was then extracted with 20 liters of 60% aqueous solution of ethanol at 40° C. with stirring until the absorbance of the 1/200 dilution at 313 nm became 0.22. The resulting extract was concentrated and freeze-dried to give 70 g (yield based on dry leaves: 7%) of freeze-dried powder of Hydrangeae Dulcis Folium from the residue of a water extract.

### EXAMPLE 6

Production of Freeze-Dried Powder an Acetone Extract of Hydrangeae Dulcis Folium Water-Extract Residue

Dried powder of Hydrangeae Dulcis Folium (1 kg) was extracted with 20 liters of distilled water at 40° C. with stirring until the absorbance of the 1/200 dilution at 313 nm became 0.15. The obtained extract was filtered to remove the filtrate. The resulting extract residue was then extracted with 20 liters of acetone at 40° C. with stirring until the absorbance of the 1/200 dilution at 313 nm became 0.22. The resulting extract was concentrated and freeze-dried to give 60 g (yield based on dry leaves: 6%) of freeze-dried powder of Hydrangeae Dulcis Folium from the water extract residue.

### EXAMPLE 7

Effect of Extract of Hydrangeae Dulcis Folium on Rats Suffering From Streptococcal Cell Wall-Induced Arthritis

It is known that arthritis is induced in Lewis rats by administering streptococcal cell wall two times [Infection & Immunity, 59(12), 4436 (1991)].

As the first administration of streptococcal cell wall, a peptidoglycan polysaccharide 100P fraction (hereinafter referred to as PG-PS 100) (LEE LABORATORIES), which was diluted with sterilized PBS (−) (9 g of sodiumchloride; 0.795 g of disodium hydrogenphosphate heptahydrate; 0.144 g of potassium dihydrogenphosphate; dissolved in 1 L of distilled water) to 0.6 mg/mL, and destroyed by ultrasonication (US-1 type; made by SND) for 20 minutes, was administered to female Lewis rats (Charles River) of 8 weeks of age at the heel joint of the right hind-limb in a dose of 10 μL (6 μg of PG-PS 100) per rat. On the other hand, sterilized PBS (−) was administered to the rats into the heel joint of the left hind-limb in a dose of 10 μL per rat as a negative control.

At day 21 after the first administration of streptococcal cell wall, as the 2nd administration of streptococcal cell wall, PG-PS 100P, which was similarly diluted with sterilized PBS (−) to 0.2 mg/mL and destroyed by ultrasonication for 20 minutes, was intravenously administered to the rats at the tail vein for sensitization in a dose of 500 μL/rat (100 μL of PG-PS 100). Thus, arthritis was induced in rats.

From 3 days before the first administration of streptococcal cell wall, 1 ml/100 g body weight of 0.5% methylcellulose 400 (Nacalai Tesque) as a control, and 1 ml/100 g body weight of a solution of 30 mg of the ethanol extract from the water-extract residue of Hydrangeae Dulcis Folium (referred to as Ethanol extract of Hydrangeae Dulcis Folium water-extract residue in Table 1) prepared in Example 5 suspended in 0.5% methylcellulose 400 respectively were administered orally to rats 5 times a week. At day 24 after the first administration of streptococcal cell wall, the edema occurring on the joint was measured with a plethysmometer (UNICOM).

In the respective conditions for treatment, 6 rats were used and a change of the edema volume in the joint was calculated from the following formula:

Change of the edema volume in the joint=(A−B)−(C−D)

A: Volume of the heel joint of the right hind-limb at day 24

B: Volume of the heel joint of the right hind-limb at day 0

C: Volume of the heel joint of the left hind-limb at day

D: Volume of the heel joint of the left hind-limb at day

Table 1 shows the results.

In Table 1, the figures indicate the change of edema volume in the joint in the respective conditions for treatment by the mean value±standard error (n=6).

In Table 1, “Untreated” indicates that no streptococcal cell wall has been administered and “Control” indicates that only 0.5% methylcellulose 400 has been orally administered.

TABLE 1

Change of the edema
volume in joint (ml)

| Treatment | After 0 day | After 24 days |
|---|---|---|
| Untreated | 0.00 ± 0.00 | 0.01 ± 0.01 |
| Control | 0.00 ± 0.00 | 0.48 ± 0.11 |
| Ethanol extract of | 0.00 ± 0.00 | 0.16 ± 0.04 |
| Hydrangeae Dulcis | | |
| Folium water-extract | | |
| residue | | |

As shown in Table 1, the volume increase in the heel joint of hind-limb, which indicates the degree of joint edema, was inhibited markedly by administration of the ethanol extract of Hydrangeae Dulcis Folium water-extract residue in comparison with that by administration of 0.5% methylcellulose 400 alone (control).

The results indicate that the administration of the ethanol extract of Hydrangeae Dulcis Folium water-extract residue effectively prevents arthritis.

### EXAMPLE 8

Production of an Agent Containing the Ethanol Extract of Hydrangeae Dulcis Folium Water-Extract Residue

The following ingredients were mixed to prepare an agent for preventing or treating arthritis.

| | | |
|---|---|---|
| Freeze-dried powder prepared in Example 5 | 49 | g |
| Pinedex #3 (Matsutani Chemical Industry) | 49 | g |
| Ferric pyrophosphate (iron source; Kokusan | 0.1 | g |
| Chemical Co., Ltd.) | | |
| Phoscal EFC (Calcium source; Nikko Fine Products) | 1 | g |
| Vitamin mixture (Merck & Co., Inc.) | 1 | g |

### EXAMPLE 9

Production of a Drink Containing the Ethanol Extract of Hydrangeae Dulcis Folium Water-Extract Residue

Into 180 ml of water was dispersed 20 g of an agent for preventing or treating arthritis prepared in Example 8 to give a drink for prevention or treatment of arthritis.

### EXAMPLE 10

Production of Cookies Containing the Ethanol Extract of Hydrangeae Dulcis Folium Water-Extract Residue

Cookies (30 pieces) were prepared from the following ingredients.

| | | |
|---|---|---|
| Soft flour | 100 | g |
| Starch | 74 | g |
| Water | 14 | g |
| Freeze-dried powder prepared | 30 | g |
| in Example 5 | | |
| Baking powder | 2 | Tsp. |
| Salt | 2 | Tsp. |
| Egg | 1 | egg |
| Butter | 80 | g |
| Milk | 2 | Tbsp. |

### EXAMPLE 11

Production of a Feed Containing 1% of the Freeze-Dried Powder of Example 1

The following feed was prepared.

| | | |
|---|---|---|
| Sucrose (Kishida Chemical Co., Ltd.) | 20.0 | wt % |
| Corn oil (Nacalai Tesque, Inc.) | 5.0 | wt % |
| Choline bitartrate (Tokyo Kasei Kogyo Co., Ltd.) | 0.4 | wt % |
| Corn starch (Nippon Starch Chemical Co., Ltd.) | 39.1 | wt % |
| AIN-76 vitamin (Oriental Yeast Co., Ltd.) | 1.0 | wt % |
| AIN-76 mineral (Oriental Yeast Co., Ltd.) | 3.5 | wt % |
| Cellulose (Oriental Yeast Co., Ltd.) | 5.0 | wt % |
| Casein (Wako Pure Chemical Industries, Ltd.) | 25.0 | wt % |
| Freeze-dried powder prepared in Example 1 | 1.0 | wt % |

### EXAMPLE 12

Production of Freeze-Dried Powder From a Water Extract of Saxifraga stolonifera Meerb.

Dried powder of Saxifraga stolonifera Meerb. (1 kg, Shihira Shoten) was extracted twice with 10 liters of distilled water at room temperature with stirring for 1 hour. The resulting extract was concentrated and freeze-dried to give 150 g of freeze-dried powder of Saxifraga stolonifera Meerb. as the water extract.

### EXAMPLE 13

Production of Freeze-Dried Powder From a 60% Ethanol-Water Extract of Saxifraga stolonifera Meerb.

Dried powder of Saxifraga stolonifera Meerb. (1 kg) was extracted with 20 liters of 60% ethanol-water at 40° C. with stirring. The resulting extract was concentrated and freeze-dried to give 315 g of freeze-dried powder of Saxifraga stolonifera Meerb. as the ethanol extract (yield based on dry leaves: 31.5%).

### EXAMPLE 14

Production of Freeze-Dried Powder From an Acetone Extract of Saxifraga stolonifera Meerb.

Dried powder of Saxifraga stolonifera Meerb. (1 kg) was extracted twice with 10 liters of acetone at room temperature with stirring for 1 hour. The resulting extract was concentrated and freeze-dried to give 164 g of freeze-dried powder of Saxifraga stolonifera Meerb. as the acetone extract.

### EXAMPLE 15

Production of freeze-dried powder from a Hot Water Extract of Saxifraga stolonifera Meerb.

Dried powder of from Saxifraga stolonifera Meerb. (1 kg) was extracted with 10 liters of boiling distilled water for 30 minutes. The resulting extract was concentrated and freeze-dried to give 100 g of freeze-dried powder of Saxifraga stolonifera Meerb. as the hot water extract.

### EXAMPLE 16

Production of Freeze-Dried Powder From an Ethanol Extract of Saxifraga stolonifera Meerb. Water-Extract Residue

Dried powder of Saxifraga stolonifera Meerb. (1 kg) was extracted with 20 liters of distilled water 40° C. with stirring. The extract was filtered to remove the filtrate, and an extract residue was obtained. The residue was extracted with 20 liters of 60% aqueous solution of ethanol at 40° C. with stirring. The resulting extract was concentrated and freeze-dried to give 80 g of freeze-dried powder of Saxifraga stolonifera Meerb. as the ethanol extract from the water extraction residue.

### EXAMPLE 17

Production of Freeze-Dried Powder From an Acetone Extract of Saxifraga stolonifera Meerb. Water-Extract Residue

Dried powder of Saxifraga stolonifera Meerb. (1 kg) was extracted with 20 liters of distilled water at 40° C. with stirring. The extract was filtered to remove the filtrate, and an extract residue was obtained. The residue was extracted with 20 liters of acetone at 40° C. with stirring. The resulting extract was concentrated and freeze-dried to give 90 g of freeze-dried powder of Saxifraga stolonifera Meerb. as the acetone extract from the water extraction residue.

### EXAMPLE 18

Production of an Agent Containing the Ethanol Extract of Saxifraga stolonifera Meerb.

The following ingredients were mixed to prepare an agent for preventing or treating arthritis.

| | | |
|---|---|---|
| Freeze-dried powder prepared in Example 13 | 49 | g |
| Pinedex #3 (Matsutani Chemical Industry) | 49 | g |
| Ferric pyrophosphate (iron source; | 0.1 | g |
| Kokusan Chemical Co., Ltd.) | | |
| Phoscal EFC (Calcium source; Nikko Fine Products) | 1 | g |
| Vitamin mixture (Merck & Co., Inc.) | 1 | g |

### EXAMPLE 19

Production of a Drink Containing the Ethanol Extract of Saxifraga stolonifera Meerb.

The agent for preventing or treating arthritis prepared in Example 18 (20 g) was dispersed in 180 ml of water to give a drink for preventing or treating arthritis.

### EXAMPLE 20

Production of Cookies Containing the Ethanol Extract of Saxifraga stolonifera Meerb.

Cookies (30 pieces) were prepared from the following ingredients.

| | | |
|---|---|---|
| Soft flour | 100 | g |
| Starch | 74 | g |
| Water | 14 | g |
| Freeze-dried powder | 30 | g |
| prepared in Example 13 | | |
| Baking powder | 2 | Tsp. |
| Salt | 2 | Tsp. |
| Egg | 1 | egg |
| Butter | 80 | g |
| Milk | 2 | Tbsp. |

### EXAMPLE 21

Production of a Feed Containing 1% by Weight of the Ethanol Extract of Saxifraga stolonifera Meerb.

The feed having the following composition was prepared.

| | | |
|---|---|---|
| Sucrose (Kishida Chemical Co., Ltd.) | 20.0 | wt % |
| Corn oil (Nacalai Tesque, Inc.) | 5.0 | wt % |
| Choline bitartrate (Tokyo Kasei Kogyo Co., Ltd.) | 0.4 | wt % |
| Corn starch (Nippon Starch Chemical Co., Ltd.) | 39.1 | wt % |
| AIN-76 vitamin (Oriental Yeast Co., Ltd.) | 1.0 | wt % |
| AIN-76 mineral (Oriental Yeast Co., Ltd.) | 3.5 | wt % |
| Cellulose (Oriental Yeast Co., Ltd.) | 5.0 | wt % |
| Casein (Wako Pure Chemical Industries, Ltd.) | 25.0 | wt % |
| Freeze-dried powder prepared in Example 13 | 1.0 | wt % |

### EXAMPLE 22

Production of a Feed Containing the Ethanol Extract of Saxifraga stolonifera Meerb.

The feed having the following composition was prepared.

| | | |
|---|---|---|
| CE-2 (Clea Japan, Inc.) | 98.0 | wt % |
| Freeze-dried powder prepared in Example 13 | 2.0 | wt % |

### EXAMPLE 23

Effect of the Extract of Saxifraga stolonifera Meerb. on Murine Arthritis Induced by Type-2 Collagen

In this test, DBA/1J mice (Charles River) were employed, in which arthritis was induced by twice administering type-2 collagen.

As the first administration of type-2 collagen, an equal mixture of type-2 collagen (Collagen Technical Training Institute) and Freund's complete adjuvant (Iatron, Inc.) emulsified with a homogenizer was administered intracutaneously to a male DBA/1J mouse of 6 weeks old at a dose of 100 μl/mouse.

At day 21 after the first administration of type-2 collagen, as the 2nd administration of type-2 collagen, similarly to the first administration, an equal mixture of type-2 collagen and Freund's complete adjuvant emulsified with a homogenizer was administered intracutaneously at a dose of 100 μl/mouse. Thus, arthritis was induced in mice.

Starting on the day of the first administration of type-2 collagen, mice were fed with a feed prepared in Example 22 containing the ethanol extract of Saxifraga stolonifera Meerb. (referred to as “Ethanol extract of Saxifraga stolonifera Meerb.” in Table 3). In a control group, mice were fed with powdered feed CE-2 containing nothing. At the days 26, 30, 34, 37 and 42 after the first administration of type-2 collagen, the degree of incidence of arthritis were scored according to the following indices.

Scoring was carried out by applying 0-4 points with respect to one of four paws of the mouse depending on the conditions as shown in Table 2, and then the total score of 4 paws of the mouse, that is, 0 to 16, was recorded. For each condition, 20 mice were subjected to observation.

TABLE 2

| Condition | Score |
|---|---|
| No swelling observed on digits and hands (feet) | 0 |
| (No symptom) | |
| Light swelling observed on one finger or on ankle | 1 |
| Swelling observed on 1 to 3 fingers or on ankle | 2 |
| Swelling observed on 3 to 5 fingers and on ankle | 3 |
| Swelling observed on all fingers and on ankles | 4 |

Table 3 shows the results. The fingers in the table indicate the scores in the respective conditions of treatment by the mean value±standard error (N=20). A statistical significant difference was carried out by a Student's In the table, “No treatment” means that the mice have with a commercially available CE-2 and no treatment e-2 collagen has been made.

TABLE 3

| | Score | | | |
|---|---|---|---|---|
| | | | Ethanol | Significant |
| Days | No treatment | Control | Ext.* | difference |
| 0 | 0.0 ± 0.0 | 0.0 ± 0.0 | 0.0 ± 0.0 | — |
| 26 | 0.0 ± 0.0 | 1.4 ± 0.4 | 1.0 ± 0.4 | — |
| 30 | 0.0 ± 0.0 | 10.4 ± 0.7 | 7.8 ± 0.9 | p = 0.026 |
| 34 | 0.0 ± 0.0 | 12.4 ± 0.5 | 9.0 ± 1.0 | p = 0.004 |
| 37 | 0.0 ± 0.0 | 12.2 ± 0.5 | 9.2 ± 0.9 | p = 0.010 |
| 42 | 0.0 ± 0.0 | 12.0 ± 0.5 | 9.3 ± 1.0 | p = 0.019 |

*Ethanol extract of Saxifraga stolonifera Meerb.

As shown in Table 3, the score of the mice, which were fed with a feed containing 2 wt % of ethanol extract of Saxifraga stolonifera Meerb. was significantly lower than that of the mice (control) which were fed with a powdered feed containing no ethanol extract of Saxifraga stolonifera Meerb., showing that the addition of the ethanol extract of Saxifraga stolonifera Meerb. can inhibit increase of the score of arthritis.

From the above results, it is proved that the incidence of arthritis can be inhibited by previously taking the extract of Saxifraga stolonifera Meerb. before incidence of arthritis.

### INDUSTRIAL APPLICABILITY

According to the present invention, it is possible to provide an agent for preventing or treating arthritis, a food and drink or feed having an effect for preventing or treating arthritis, an additive to foods and drinks or feeds having an effect for preventing or treating arthritis, and a method for preventing or treating arthritis in animals.

1. An agent of preventing or treating arthritis which comprises a plant belonging to the family Saxifragaceae or its extract as an active ingredient.

2. An agent for preventing or treating arthritis according to claim 1, wherein the plant belonging to the family Saxifragaceae belongs to the genus Saxifraga.

3. An agent for preventing or treating arthritis according to claim 2, wherein the plant belonging to the genus Saxifraga is Saxifraga stolonifera Meerb.

4. An agent for preventing or treating arthritis according to claim 1, wherein the plant belonging to the family Saxifragaceae belongs to the genus Hydrangea.

5. An agent for preventing or treating arthritis according to claim 4, wherein the plant belonging to the genus Hydrangea is Hydrangea macrophylla Seringe var. Thunbergii Makino.

6. An agent for preventing or treating arthritis according to any of claims 1 to 5, wherein the extract of the plant belonging to the family Saxifragaceae is an alcohol extract of the residue of the extract extracted with an aqueous medium.

7. An agent for preventing or treating arthritis according to any of claims 1 to 6, wherein the arthritis is chronic articular rheumatism.

8. An agent for preventing or treating arthritis according to any of claims 1 to 7, which is administered orally.

9. A food and drink which comprises a plant belonging to the family Saxifragaceae or its extract.

10. A food and drink according to claim 9, which is used for preventing or treating arthritis.

11. A food and drink according to claims 9 or 10, wherein the plant belonging to the family Saxifragaceae belongs to the genus Saxifraga.

12. A food and drink according to claim 11, wherein the plant belonging to the genus Saxifraga is Saxifraga stolonifera Meerb.

13. A food and drink according to claims 9 or 10, wherein the plant belonging to the family Saxifragaceae belongs to the genus Hydrangea.

14. A food and drink according to claim 13, wherein the plant belonging to the genus Hydrangea is Hydrangea macrophylla Seringe var. Thunbergii Makino.

15. A food and drink according to any of claims 9 to 14, wherein the extract of the plant belonging to the family Saxifragaceae is an alcohol extract of the residue of the extract extracted with an aqueous medium.

16. A feed which comprises a plant belonging to the family Saxifragaceae or its extract.

17. A feed according to claim 16, which is used for preventing or treating arthritis.

18. A feed according to claims 16 or 17, wherein the plant belonging to the family Saxifragaceae belongs to the genus Saxifraga.

19. A feed according to claim 18, wherein the plant belonging to the genus Saxifraga is Saxifraga stolonifera Meerb.

20. A feed according to claims 16 or 17, wherein the plant belonging to the family Saxifragaceae belongs to the genus Hydrangea.

21. A feed according to claim 20, wherein the plant belonging to the genus Hydrangea is Hydrangea macrophylla Seringe var. Thunbergii Makino.

22. A feed according to any of claims 16 to 21, wherein the extract of the plant belonging to the family Saxifragaceae is an alcohol extract of the residue of the extract extracted with an aqueous medium.

23. An additive for foods and drinks having an effect to prevent or treat arthritis, which comprises a plant belonging to the family Saxifragaceae or its extract.

24. An additive for foods and drinks according to claim 23, wherein the plant belonging to the family Saxifragaceae belongs to the genus Saxifraga.

25. An additive for foods and drinks according to claim 24, wherein the plant belonging to the genus Saxifraga is Saxifraga stolonifera Meerb.

26. An additive for foods and drinks according to claim 23, wherein the plant belonging to the family Saxifragaceae belongs to the genus Hydrangea.

27. An additive for foods and drinks according to claim 26, wherein the plant belonging to the genus Hydrangea is Hydrangea macrophylla Seringe var. Thunbergii Makino.

28. An additive for foods and drinks according to any of claims 23 to 27, wherein the extract of the plant belonging to the family Saxifragaceae is an alcohol extract of the residue of the extract extracted with an aqueous medium.

29. An additive for feeds having an effect to prevent or treat arthritis, which comprises a plant belonging to the family Saxifragaceae or its extract.

30. An additive for feeds according to claim 29, wherein the plant belonging to the family Saxifragaceae belongs to the genus Saxifraga.

31. An additive for feeds according to claim 30, wherein the plant belonging to the genus Saxifraga is Saxifraga stolonifera Meerb.

32. An additive for feeds according to claim 29, wherein the plant belonging to the family Saxifragaceae belongs to the genus Hydrangea.

33. An additive for feeds according to claim 32, wherein the plant belonging to the genus Hydrangea is Hydrangea macrophylla Seringe var. Thunbergii Makino.

34. An additive for feeds according to any of claims 29 to 33, wherein the extract of the plant belonging to the family Saxifragaceae is an alcohol extract of the residue of the extract extracted with an aqueous medium.

35. A method for preventing or treating arthritis in animals which comprises administering an agent for preventing or treating arthritis according to any of claims 1 to 7 to an animal or feeding a feed according to any of claims 16 to 22 to an animal.

36. A method according to claim 35, wherein the animal is a pet or domestic animal.

37. A food and drink or feed for preventing or treating arthritis, which comprises a plant belonging to the family Saxifragaceae or its extract as an active ingredient.

38. A food and drink or feed for preventing or treating arthritis according to claim 37, wherein the plant belonging to the family Saxifragaceae belongs to the genus Saxifraga.

39. A food and drink or feed for preventing or treating arthritis according to claim 38, wherein the plant belonging to the genus Saxifraga is Saxifraga stolonifera Meerb.

40. A food and drink or feed for preventing or treating arthritis according to claim 37, wherein the plant belonging to the family Saxifragaceae belongs to the genus Hydrangea.

41. A food and drink or feed for preventing or treating arthritis according to claim 40, wherein the plant belonging to the genus Hydrangea is Hydrangea macrophylla Seringe var. Thunbergii Makino.

42. A food and drink or feed for preventing or treating arthritis according to any of claims 37 to 41, where in the extract of the plant belonging to the family Saxifragaceae is an alcohol extract of the residue of the extract extracted with an aqueous medium.

43. Use of a plant belonging to the family Saxifragaceae or its extract for the production of an agent for preventing or treating arthritis.

44. Use of a plant belonging to the family Saxifragaceae or its extract for the production of a food and drink or feed for preventing or treating arthritis.

45. Use of a plant belonging to the family Saxifragaceae or its extract for the production of an additive for a food and drink or feed for preventing or treating arthritis.

46. A method for preventing or treating arthritis which comprises administering a plant belonging to the family Saxifragaceae or its extract.
